(19) United States
(12) Patent Application Publication
(10) Pub. No.: US 2021/0267688 A1
(43) Pub. Date: Sep. 2, 2021

(54) SYSTEMS AND METHODS FOR SIMULATION AND MODELING OF COMBINED AUGMENTATION PROCEDURES

(71) Applicant: Establishment Labs S.A., La Garita, Alajuela (CR)

(72) Inventors: Juan José CHACÓN QUIRÓS, Alajuela (CR); Marcos SFORZA, Birmingham (GB); Rafael G. CORRALES, San José (CR); Raghu RAGHAVAN, Baltimore, MD (US); Roberto DE MEZERVILLE, Alajuela (CR); Rudy A. MAZZOCCHI, New York, NY (US); Martin BRADY, Baltimore, MD (US); Gerardo MORA, Alajuela (CR); Gerson RODRIGUEZ, Alajuela (CR)

(73) Assignee: Establishment Labs S.A., La Garita, Alajuela (CR)

(21) Appl. No.: 17/254,760

(22) PCT Filed: Jun. 21, 2019
(86) PCT No.: PCT/US2019/038536

§ 371 Date: Dec. 21, 2020

Related U.S. Application Data
(60) Provisional application No. 62/688,778, filed on Jun. 22, 2018.

(51) Int. Cl. A61B 34/10; G16H 50/50; G16H 20/40; G16H 50/70; G16H 40/20; G16H 50/30; G16H 30/20; G06F 30/20; G06F 3/0484

(52) U.S. Cl. CPC A61B 34/10; G16H 50/50; G16H 20/40; G16H 50/70; A61F 2/12; G16H 50/30; G16H 30/20; G06F 30/20; G06F 3/04847; G16H 40/20

(57) ABSTRACT

Systems and methods for devising and/or simulating surgical augmentation procedures including combinations of implants and secondary materials are disclosed herein. An exemplary method includes receiving parameters for a pre-operative state of an implantation site, receiving parameters for a post-operative state of the implantation site, automatically generating a hybrid strategy for achieving the post-operative state from the preoperative state, wherein the hybrid strategy includes a proposed implant volume and a proposed volume of a secondary material, and generating a simulation of the post-operative state of the implantation site using the proposed implant volume and the proposed volume of the secondary material.

### CROSS-REFERENCE TO RELATED APPLICATIONS

This application claims priority to U.S. Provisional Application No. 62/688,778, filed on Jun. 22, 2018, which is incorporated by reference herein in its entirety.

### TECHNICAL FIELD

The present disclosure relates to systems and methods useful for medical procedures, such as, e.g., aesthetic and/or reconstructive surgeries.

### BACKGROUND

Aesthetic, cosmetic, and reconstructive surgeries refer to surgeries performed in order to repair, restore, or change the appearance of a subject's anatomy. For example, the field of cosmetic surgery includes surgeries such rhytidectomy (facelifts), mammoplasty (changing the size of the breasts), and gluteoplasty (changing the size of the buttocks), and the field of reconstructive surgery includes implanting a prosthesis and procedures such as the reattachment of an amputated body part. In some such procedures, a surgeon inserts a suitable implant at a desired region of the subject's body. In some cases, an implant alone may not provide a desired size, shape, or change in physical appearance or feel of the subject's body part. Additionally, an implant alone may have an undesirable weight or feel to a subject. Moreover, in some cases, the subject may have to wait for the conclusion of the procedure to visualize the results of the procedure.

### SUMMARY

Systems and methods for simulating an outcome of a surgical procedure are disclosed herein. In some aspects, a method for simulating an outcome of a surgical procedure includes receiving parameters for a pre-operative state of an implantation site, receiving parameters for a post-operative state of the implantation site, based on the parameters for the pre-operative and post-operative states, automatically generating a hybrid strategy for achieving the post-operative state from the pre-operative state, wherein the hybrid strategy includes a proposed implant volume and a proposed volume of a secondary material, and generating a simulation of the post-operative state of the implantation site using the proposed implant volume and the proposed volume of the secondary material.

Receiving parameters for the post-operative state of the implantation site may include providing a catalog of potential implants for use at the implantation site, and receiving a selection of an initial implant from the catalog of potential implants. For example, the parameters for the pre-operative state of the implantation site may include a pre-operative volume, and the parameters for the post-operative state of the implantation site may include a post-operative volume.

According to some aspects of the present disclosure, automatically generating the hybrid strategy includes calculating a difference in volume between the post-operative state and the pre-operative state, determining the proposed implant volume by applying a skin quality coefficient to the difference in volume, and determining the proposed volume of the secondary material by subtracting the proposed implant volume from the difference in volume. In some aspects, a volume of the post-operative state may be less than or equal to twice a volume of the pre-operative state, wherein determining the proposed implant volume further includes applying the skin quality coefficient to the volume difference to obtain a first value, and applying the skin quality coefficient to the first value to obtain the proposed implant volume. For example, the skin quality coefficient may be between about 0.5 and about 0.6. Further, according to some aspects, determining the proposed volume of the secondary material includes factoring in a reabsorption rate of the secondary material. The secondary material may comprise, for example, fat and/or a synthetic filler.

The present disclosure also includes a method for simulating an outcome of a surgical procedure that comprises receiving parameters for a pre-operative state of an implantation site, receiving a selection of an initial implant from a digital catalog, generating a first visual simulation of the implantation site in a first post-operative state, wherein the first post-operative state includes the selected initial implant, generating a second visual simulation of the implantation site in a second post-operative state, wherein the second post-operative state includes a second implant, optionally selected from a digital catalog or database, and a volume of secondary material, receiving an input adjusting a parameter of the second post-operative state, and generating an adjusted second visual simulation of the implantation site in the second post-operative state to account for the adjusted parameter.

The adjusted parameter may include a change in a distribution of the volume of secondary material at the implantation site. Further, for example, the input adjusting the parameter of the second post-operative state may include a third implant different from each of the initial implant and the second implant. The steps of receiving parameters for a pre-operative state of an implantation site, receiving selection of an initial implant from a digital catalog, and receiving an input adjusting a parameter of the second post-operative state may include receiving data from a graphical user interface.

According to some aspects of the present disclosure, the second visual simulation includes a distribution of the volume of secondary material in one or more quadrants of the implantation site, and receiving the input adjusting the parameter of the second post-operative state includes receiving an indicated amount of secondary material for addition to or subtraction from at least one quadrant of the one or more quadrants of the implantation site. The method may further include displaying a side-by-side view of the first visual simulation and the second visual simulation. In some examples, receiving parameters for the pre-operative state of the implantation site includes receiving digital imaging data of the implantation site.

In some aspects of the present disclosure, there is a method for simulating an outcome of a surgical procedure that includes receiving parameters for a pre-operative state of an implantation site, generating a simulation of the implantation site using the parameters for the pre-operative state and a proposed implant volume, receiving placement parameters for a volume of secondary material to be added to the implantation site, and modifying the simulation of the implantation site to include the volume of secondary material. Generating the simulation of the implantation site may include, for example, dividing the implantation site into segments. In some examples, the placement parameters for the volume of secondary material includes identification of a segment for placement of the volume of secondary material.

Further, for example, modifying the simulation of the implantation site includes increasing a volume of the identified segment corresponding to the volume of secondary material, and generating a resulting displacement at a plurality of points on a perimeter of the implantation site, wherein a magnitude of displacement for each point of the plurality of points negatively correlates to a distance between the identified segment and the point. The proposed implant volume may correspond to an implant from a catalog of potential implants. As mentioned above, the secondary material optionally may comprise fat or a synthetic filler.

### BRIEF DESCRIPTION OF THE DRAWINGS

The accompanying drawings, which are incorporated in and constitute a part of this specification, illustrate exemplary embodiments of the present disclosure. In these drawings, where appropriate, reference numerals illustrating similar elements are labeled similarly. For simplicity and clarity of illustration, the figures depict the general structure and/or manner of construction of the various embodiments. Descriptions and details of well-known features and techniques may be omitted to avoid obscuring other features. Elements in the figures are not necessarily drawn to scale. The dimensions of some features may be exaggerated relative to other features to improve understanding of the exemplary embodiments. For example, one of ordinary skill in the art will appreciate that some views may not be drawn to scale. Further, even if it is not specifically mentioned in the text, aspects described with reference to one embodiment may also be applicable to, and may be used with, other embodiments.

FIG. 1 depicts, in flow chart form, an exemplary method according to some aspects of the present disclosure.

FIG. 2 depicts, in flow chart form, another exemplary method according to some aspects of the present disclosure.

FIG. 3 depicts, in flow chart form, another exemplary method according to some aspects of the present disclosure.

FIG. 4 depicts, in flow chart form, another exemplary method according to some aspects of the present disclosure.

FIGS. 5-7 depict views of an exemplary user interface according to some aspects of the present disclosure.

FIGS. 8-11 depict views of another exemplary user interface according to some aspects of the present disclosure.

FIG. 12 depicts, in flow-chart form, a further exemplary method according to some aspects of the present disclosure.

FIG. 13 depicts, in schematic form, an exemplary system according to some aspects of the present disclosure.

### DETAILED DESCRIPTION

Aspects of the present disclosure may be used to visualize physical features of a subject, such as a patient contemplating a medical procedure, and simulate changes in the subject's appearance resulting from the medical procedure. Aspects of the present disclosure may be used to simulate the results of aesthetic or reconstructive surgeries. Advantageously, aspects of the present disclosure may allow for a hybrid approach to cosmetic procedures such as breast augmentation surgery, gluteal augmentation surgery, and the like, where the hybrid approach may have improved reproducibility, improved predictability, and/or improved surgical outcomes. Aspects of the present disclosure may offer, for example, a procedure that reduces complications related to greater implant volume and the associated weight of an implant having a greater volume, and/or a better alternative for surgeons and patients who may appreciate lightweight implants, along with the capability of, e.g., not only enlarging, constructing, or reconstructing subject anatomy, but being able to sculpt a final outcome for an individual subject.

Embodiments of the present disclosure may provide one or more additional benefits, such as simulation capabilities (e.g., three-dimensional simulation capabilities) for surgeons who perform breast augmentation and other cosmetic surgeries and their patients, allowing such surgeons to model hybrid augmentation strategies combining an implant with one or more secondary materials. Moreover, projected outcomes achieved by the systems and methods disclosed herein may assist surgeons and patients by providing pre-surgical consultation recommendations and procedural guidance as to insertion/injection locations, and volume(s) of secondary material(s) to be used in augmenting an implant having a lower volume and a smaller size than would otherwise be needed to achieve a desired result.

Aspects of the present disclosure are described in greater detail below. The terms and definitions as used and clarified herein are intended to represent the meaning within the present disclosure. The terms and definitions provided herein control, if in conflict with terms and/or definitions incorporated by reference.

In the discussion that follows, relative terms such as “about,” “substantially,” “approximately,” etc. are used to indicate a possible variation of ±5% in a stated numeric value. It should be noted that the description set forth herein is merely illustrative in nature and is not intended to limit the embodiments of the subject matter, or the application and uses of such embodiments. Any implementation described herein as exemplary is not to be construed as preferred or advantageous over other implementations. Rather, the term “exemplary” is used in the sense of example or illustrative. The terms “comprise,” “include,” “have,” “with,” and any variations thereof are used synonymously to denote or describe non-exclusive inclusion. As such, a process, method, system, or device that uses such terms does not include only those steps, structure or elements but may include other steps, structures or elements not expressly listed or inherent to such process, method, system, or device. Further, terms such as “first,” “second,” and the like, if used herein, do not denote any order, quantity, or importance, but rather are used to distinguish one element from another. Similarly, terms of relative orientation, such as “front side, “top side,” “back side,” “bottom side,” “upper,” “lower,” etc., are referenced relative to the described figures.

The term “implantation site” as used herein may refer to a portion of a body (e.g., a body of a human or animal subject) where use (e.g., implantation) of an implant in a surgical procedure is being considered. For example, an implantation site according to the present disclosure may include an area of a chest, a gluteal area, a genital area, an arm, leg, hand, foot, or other limb, or any other area of a body.

The term “implant” as used herein may refer to any biocompatible implantable device designed for body contouring, such as a breast, gluteal, pectoral, penile, calf, or facial implant. Such implantable devices may be made from silicone (e.g., silicone gel), saline, plastic or other polymer(s) or copolymer(s), and/or other materials, e.g., biocompatible materials, useful in the field of aesthetics and plastic surgery.

The present disclosure generally relates to surgical procedures including the use of medical implants, including aesthetic and reconstructive surgery. Various aspects of the present disclosure may be used with and/or include one or more features disclosed in International Application No. PCT/IB2017/0000247, entitled “Transponders and Sensors for Implantable Medical Devices and Methods of Use Thereof,” filed on Feb. 8, 2017 and published as WO2017/137853; International Application No. PCT/IB2017/000380, entitled “Medical Imaging Systems, Devices, and Methods,” filed on Apr. 4, 2017, and published as WO2017/175055; International Application No. PCT/US2017/027807, entitled “Apparatus for the Implantation of Medical Devices and Methods of Use Thereof,” filed on Apr. 14, 2017; International Application No. PCT/US2017/031948, entitled “Medical Implants and Methods of Preparation Thereof,” filed on May 10, 2017, and published as WO2017/196973; U.S. Application Publication No. 2015/0282926; U.S. Application Publication No. 2014/0081398; and/or U.S. Application Publication No. 2014/0078013, each incorporated herein by reference.

Methods according to the present disclosure may be performed using computing hardware and/or software. For example, one or more algorithms may be programmed on, e.g., a computer or series of computers, to automatically execute aspects of the methods disclosed herein. Additionally, in some embodiments, the present disclosure may include imaging and/or simulation systems that may be used in, or in preparation for, cosmetic surgery (or another medical procedure). The systems may be used to capture, measure, and/or calculate a subject's pre-operative state, and/or visualize and/or simulate expected changes in a subject's appearance resulting from a contemplated medical procedure. Additionally, the systems may be used in conjunction with data storage devices (e.g., computer storage, cloud storage, and/or databases) housing data identifying implants, secondary materials, and their characteristics, in order to identify or suggest implants, secondary materials, potential implant sizes, and/or volumes or quantities of secondary materials for use in a surgery. Moreover, systems disclosed herein may be able to simulate the use of suggested implants and/or secondary materials at an implantation site.

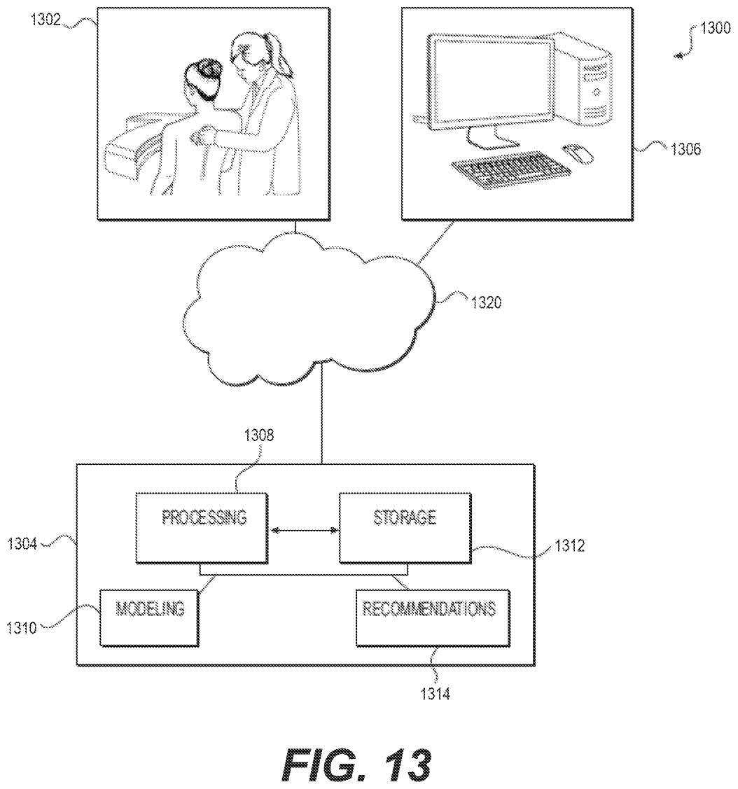

An exemplary system including imaging, modeling, recommendation, computing, and user interface components is described herein with respect to FIG. 13, discussed below. Such an exemplary system or parts thereof may be used to perform all or part of any method disclosed herein. It will be understood by one of ordinary skill in the art that any suitable computing system, imaging system, and/or user interface device may be programmed or adapted to perform part or all of the methods disclosed herein.

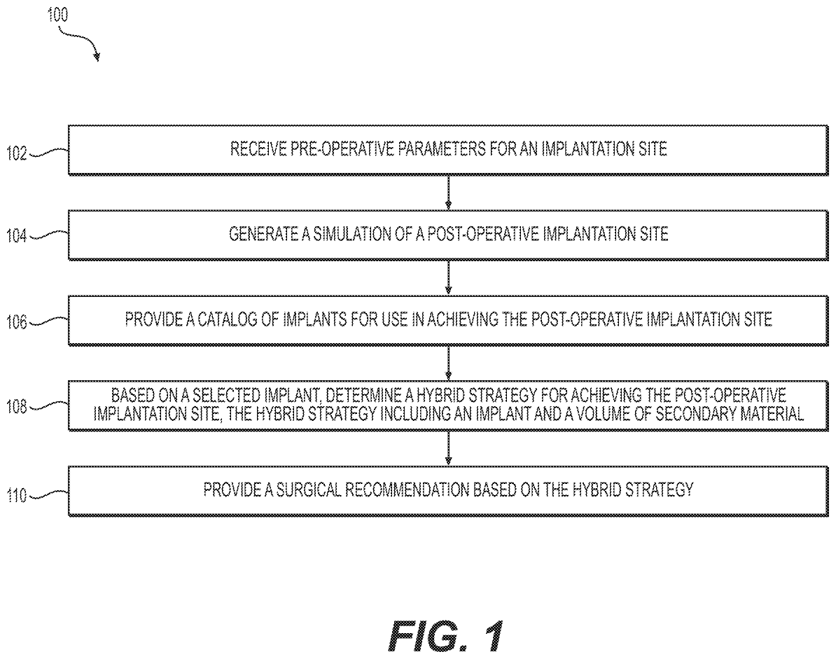

FIG. 1 illustrates an exemplary method 100 according to aspects of the present disclosure. Method 100 may include receiving pre-operative parameters for an implantation site (step 102), generating a simulation of a post-operative implantation site (step 104), providing a catalog or database of potential implants for use in achieving the post-operative implantation site (step 106), based on a selected implant, determining a hybrid strategy for achieving the target post-operative implantation site, the hybrid strategy including an implant and a volume of secondary material (step 108), and providing a surgical recommendation based on the hybrid strategy (step 110).

Generally, method 100 may result in generation of one or more simulations of an implantation site, and may use input information (e.g., selection of an implant by a user) to determine and output a recommended hybrid strategy for a surgical procedure involving an implant. The hybrid strategy may include both an implant volume (also referred to herein as a first volume) and a volume of secondary material (also referred to herein as a second volume), and may be tailored to achieve a particular result at an implantation site. In some embodiments, the hybrid strategy may be configured to provide a naturalistic, visually and tactilely smooth, and/or comfortable post-operative result at an implantation site.

Receiving pre-operative parameters for an implantation site according to step 102 may include receiving information to assist in simulating, preparing for, or performing a surgery involving an implant. For example, pre-operative parameters may include, e.g., measurements of an implantation site, data regarding the individual whose body includes the implantation site (e.g., the subject), such as demographic data, age, sex, gender, height, weight, physical conditions, reasons for wanting or needing a surgical procedure, prior surgical procedures, etc. In some embodiments, pre-operative parameters may include characteristics of an implantation site on a subject's body, such as skin quality (e.g., laxity), tissue health, prior surgical history, or other characteristics. In some embodiments, pre-operative parameters may include a series of dimensions of an implantation site, such as the depth of an incision, incision width and/or length, incision location, and/or a volume of tissue at or proximate the implantation site. In some embodiments, pre-operative parameters may include images taken by one or more imaging devices (e.g., a scanner, camera, etc.). For example, pre-operative parameters may be collected using high-resolution scanning, ultrasound imaging, high-resolution photography, three-dimensional imaging, or visual inspection, among other methods. In some embodiments, pre-operative parameters may include images taken using devices, systems and methods disclosed in International Application Publication No. WO/2017/175055. Additionally, pre-operative parameters may be collected by examining deeper tissue properties of an implantation site and the surrounding area, using, e.g., ultrasonic elasto-graphic techniques. In other variations, measurements may be taken by Eulerian Video Magnification techniques and variations thereon. In some embodiments, pre-operative parameters may include images that have been processed to determine, e.g., one or more dimensions of an implantation site.

Generating a simulation of the post-operative implantation site according to step 104 may include, e.g., using the received pre-operative parameters in conjunction with anticipated post-operation parameters to create a visual representation of the implantation site. Post-operative parameters may include data characterizing a desired outcome of a surgical procedure at the implantation site (e.g., an augmentation procedure including an implant), such as desired measurements of a post-operative implantation site (e.g., height, width, volume, shape, and/or implant type). To generate a simulation of the post-operative implantation site, the pre-operative parameters may be used as a base or starting point from which post-operative conditions may be determined. For example, in the case of a mammoplasty, a subject's pre-operative breast size (e.g., shape and volume), in conjunction with a known shape and/or volume of a desired post-operative breast size, may be used to depict a post-operative breast size on a simulation of a subject's torso. In some embodiments, the generated simulation of the post-operative implantation site may include, e.g., a three-dimensional visual simulation which may be output to a user device and/or saved for reference or later use.

Providing a catalog or database of implants for use in achieving the post-operative implantation site according to step 106 may include processing the pre-operative parameters of the implantation site and the simulation of the post-operative implantation site to determine implants that may be used to achieve the characteristics of the post-operative implantation site. For example, to achieve a desired volume at an implantation site in a post-operative state (e.g., volume of a bodily part, such as a breast or buttock) that is greater than the volume of the pre-operative implantation site, it may be determined that implants of a particular volume range may be useful. Moreover, to achieve a desired shape, texture, weight, or compatibility with a subject's body, it may be determined that implants having particular shapes and material compositions may be appropriate.

For example, in some embodiments, round implants may be selected, whereas in others, an oval, teardrop, or other shape may be selected. As a further example, in some embodiments, implants having a fluid filling such as silicone gel or saline liquid may be selected, whereas in other embodiments, implants having a structured interior, less viscous filling material, and/or a more solid interior (including, e.g. high viscosity materials, e.g., providing a “gummy bear” interior) may be selected. In some embodiments, implants having a filling material with a viscosity and elasticity providing for gravity-sensitive characteristics may be selected. In some embodiments, implants having smooth-textured surfaces, micro-textured surfaces, rough-textured surfaces, or a combination thereof may be selected. Exemplary characteristics of implants which may be found to be suitable according to the present disclosure are disclosed in, e.g., U.S. Application Publication No. 2015/0282926, U.S. Application Publication No. 2014/0081398, and/or International Application Publication No. WO2017/196973. Providing a catalog of implants may include, e.g., providing a list, database, group of images, etc., identifying the implants available, so that one or more implants may be selected by a user. In some embodiments, providing a catalog of implants may include exporting or outputting a list, database, or group of implants to, e.g., a user device (e.g., user device 1306 depicted in FIG. 13).

In some embodiments, method 100 may include receiving a selection of an implant from the catalog of implants. Such a selection may be made automatically, e.g., by a computer system performing method 100, or may be made manually, e.g., by a physician, patient, or other individual. In some embodiments, selection of an implant may assist in, e.g., performing further steps according to method 100.

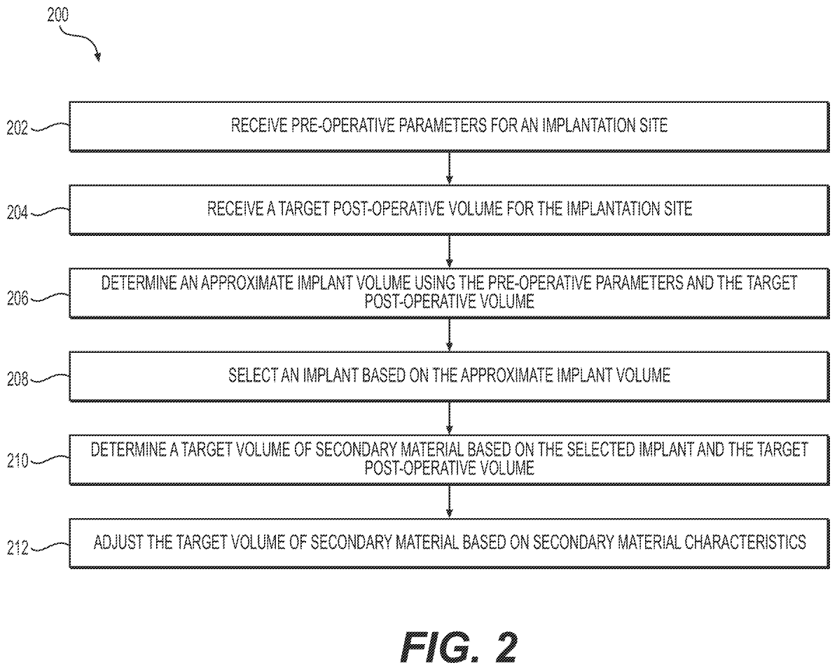

Generally, determining a hybrid strategy for achieving the target post-operative implantation site according to step 108 may include executing one or more algorithms using, e.g., pre-operative parameters of the implantation site and a simulation of the post-operative implantation site to calculate a combination of an implant and one or more secondary materials that, when implanted, injected, or otherwise added to the implantation site, aid in achieving a size (volume), a shape, and/or other characteristics of the post-operative implantation site. One such type of algorithm is described in further detail below with reference to FIG. 2. In embodiments in which an implant is selected from the catalog of implants, performing step 108 optionally may include using parameters of the selected implant, such as the volume of the selected implant, as a basis for determining the hybrid strategy. Step 108 may involve using the simulation of the post-operative implantation site and the pre-operative parameters for the implantation site to determine a difference between the pre-operative implantation site and the post-operative implantation site, such as a difference in size (volume) and/or shape. The determined difference may serve as a basis for determining a hybrid strategy. Additionally or alternatively, a target post-operative parameter (e.g., shape or volume) may serve as a basis for determining a hybrid strategy.

In some embodiments, step 108 may include identifying a target post-operative volume to be added to the implantation site (also referred to herein as a total volume), including identifying a first volume to be added as an implant, and identifying a second volume of a secondary material to be added in addition to the implant, e.g., each of the first volume and the second volume being a fraction of the total volume to be added to the implantation site. Advantageously, adding a second volume of a secondary material to an implantation site along with an implant, instead of adding an implant that comprises the entire volume to be added to the implantation site, may result in a more naturalistic, better supported, and/or customizable result from the surgical procedure.

The secondary material may be any suitable biocompatible material for injecting, implanting, or otherwise supplementing at an implantation site along with the implant. Exemplary suitable materials include, e.g., fat (such as heterologous or autologous fat), natural fillers, synthetic fillers, combinations thereof, and/or combinations of scaffolding materials useful in the field of aesthetics and cosmetic surgery. Particular advantages may vary depending on a type of a secondary material selected. For example, fat grafts may provide for a more natural result at the post-operative implantation site and/or better acceptance (biocompatibility) of an implant at the implantation site. As a further example, scaffolding materials may allow for improved structuring of a post-operative implantation site, and/or improved positioning and/or anchoring of an implant within the implantation site. Additionally, placement and division of a secondary material at an implantation site may be customizable, to provide a bespoke shape or size to a post-operative implantation site.

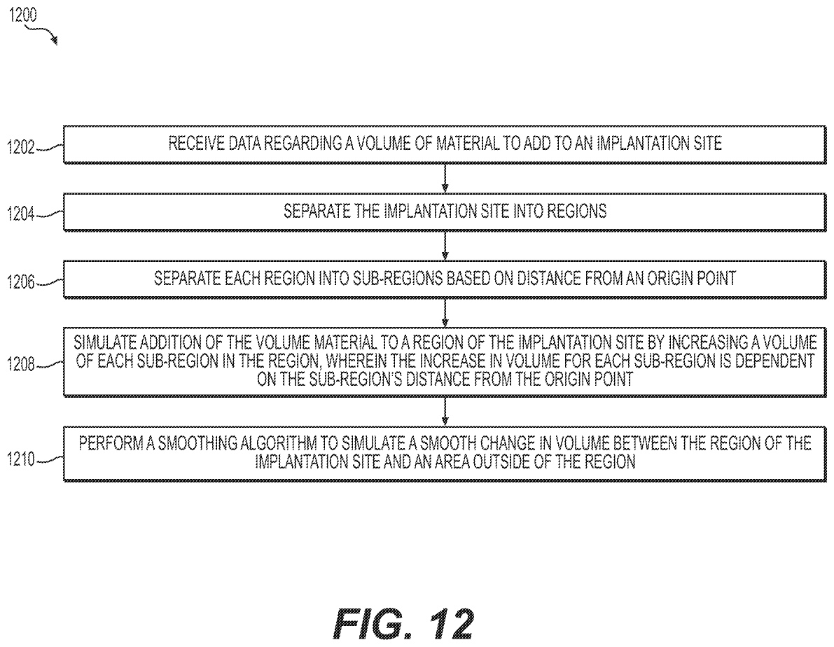

In some embodiments, in addition to determining an implant volume (first volume) and a volume of secondary material (second volume), determining a hybrid strategy according to step 108 may include determining parameters for placing the volume of secondary material at the implantation site. In some embodiments, for example, the implantation site may be divided into different regions (e.g., different segments and/or subsections). The volume of secondary material may be divided amongst different regions. In some embodiments, an algorithm may be utilized to automatically divide the volume of secondary material amongst the different regions (see, e.g., FIG. 12 and the associated discussion herein). In further embodiments, input (e.g., from a user, such as a physician or patient, or from a digital source, such as a database) may be used to determine and/or adjust the division of the volume of secondary material amongst the different regions.

As mentioned above, determining the hybrid strategy may include determining a total volume to be added to an implantation site. For example, the total volume may be determined by calculating a pre-operative implantation site volume using, e.g., pre-operative implantation parameters, calculating a post-operative implantation site volume using, e.g., parameters of the post-operative implantation site simulated in step 104, and calculating a difference between the post-operative implantation site volume and the pre-operative implantation site volume. Additionally or alternatively, the total volume may be assumed to be the volume of an initial implant selected from the catalog of implants, which may be independently selected or may be selected based on the calculated difference of post-operative implantation site volume and the pre-operative implantation site volume (e.g., an initial implant having a volume closest to the calculated difference).

In some embodiments, determining the hybrid strategy may include running through a series of “optional” scenarios (e.g., 2, 3, 4, 5, or more scenarios), which may result in one or more simulations of an implantation site reflecting an implant of predetermined dimensions and volume, supplemented with a volume of one or more secondary materials.

Providing a surgical recommendation based on the hybrid strategy according to step 110 may include selecting and outputting a hybrid strategy to, e.g., a physician, patient, digital repository, or other recipient. In some embodiments, providing a surgical recommendation may include outputting the hybrid strategy to a user interface. In some embodiments, providing the surgical recommendation may include recommending an implant shape, type, and/or volume along with a volume and type of secondary material. In some embodiments, a surgical recommendation may also include recommending an incision site, placement parameters for the volume of secondary material, and/or injection/insertion locations for the volume of secondary material or for the implant based on physical and/or biological properties of, e.g., the subject, the implant, and/or the secondary material, to enhance a likelihood of replicating a desired outcome.

FIG. 2 illustrates an exemplary method 200 according to aspects of the present disclosure. As discussed above, FIG. 2 shows in further detail steps in developing a hybrid strategy (e.g., step 108 of method 100). Method 200 may include receiving pre-operative parameters for an implantation site (step 202), receiving a target post-operative volume for the implantation site (step 204), determining an approximate implant volume using the pre-operative parameters and the target post-operative volume (step 206), selecting an implant based on the approximate implant volume (step 208), determining a target volume of secondary material based on the selected implant and the target post-operative volume (step 210), and adjusting the target volume of secondary material based on secondary material characteristics (step 212).

Receiving pre-operative parameters for an implantation site according to step 202 may include any and/or all aspects of step 102 described with respect to method 100. Receiving a target post-operative volume for the implantation site according to step 204 may include, e.g., receiving of a target post-operative volume for the implantation site from, e.g., a user, and/or may include calculating, simulating, or extrapolating a target post-operative volume. For example, step 204 may include receiving a selection of an implant (e.g., from a catalog of implants provided according to step 106 of method 100), and extrapolating a post-operative volume using the received pre-operative parameters for the implantation site and the volume of the selected implant. In some embodiments, step 204 may include generating a simulation of a post-operative implantation site which a user can adjust (by, e.g., selecting a particular implant), and then calculating a target post-operative volume using the adjusted simulation.

Determining an approximate implant volume using the pre-operative parameters and the target post-operative volume according to step 206 may include determining what fraction or percentage of the target post-operative volume (total volume) should comprise an implant. This fraction may vary based on, e.g., characteristics of the subject. For example, in some embodiments, a skin laxity of a subject may affect the selection of implant volume. For example, a subject having a higher skin laxity may desire or require that an implant make up a greater percentage (e.g., about 65%) of a target post-operative volume in order to, e.g., properly shape and support the subject's skin, as opposed to a subject having a lower skin laxity, who may desire or require that an implant make up a smaller percentage (e.g., about 55%) of a target post-operative volume. This parameter of skin laxity may be subjectively calculated using a “pinch test”, or may be determined by other suitable methods of quantifying/qualifying the amount of elasticity (elastin and/or collagen) in the skin and underlying tissue equating to laxity.

The fraction or percentage of the target post-operative volume to be accounted for by an implant may also or alternatively vary depending on, e.g., physician recommendations, patient preferences, and/or a combination thereof. In some embodiments, the percentage of the target post-operative volume to be added to the implantation site as an implant (i.e., implant volume) may vary from, e.g., about 45% to about 80%, such as from about 50% to about 70%, from about 55% to about 65%, or about 45%, about 50%, about 55%, about 60%, about 65%, about 70%, about 75%, or about 80%. In some embodiments, the percentage of the target post-operative volume to be added to the implantation site as an implant may be divided by 100 to arrive at a coefficient. In embodiments in which skin quality (e.g., skin laxity) factors into the percentage of the target post-operative volume to be added as an implant, the resulting coefficient may be referred to as a skin quality coefficient.

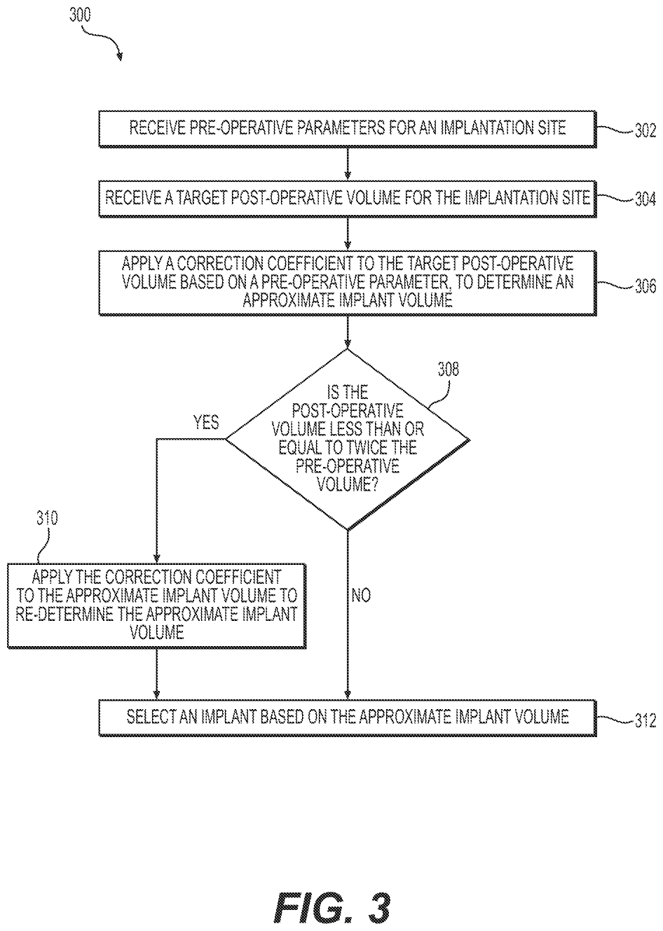

To arrive at an approximate implant volume, the target post-operative volume may be multiplied by the determined coefficient. For example, if a determined percentage of a target post-operative volume to be accounted for by an implant is 65% and the target post-operative volume is 400 cc, then the approximate implant volume may be 0.65×400 cc, or 260 cc. In some circumstances, as is described further with respect to FIG. 3, the target post-operative volume may be multiplied by the determined coefficient twice (or more times) to arrive at a suitable approximate implant volume.

Selecting an implant based on the approximate implant volume according to step 208 may include reviewing one or more catalogs or databases of available implants and selecting an implant having a volume close to the approximate implant volume. For example, an implant having a smaller or larger volume than the approximate implant volume may be selected. This may account for situations in which an implant having precisely the approximate implant volume is unavailable. Implants are often produced in a limited variety of sizes, for example. Further, multiple factors may limit the availability of implants, such as manufacturer or distributor inventory, a desired implant shape, surface texture, filling texture, viscosity, etc. In some cases, a physician may only selectively work with one or a few brands of implants, further limiting the availability of a wide variety of implant volumes.

In some embodiments, step 208 may be performed automatically; for example, a computer system performing method 200 may review one or more digital implant catalogs and may automatically select an implant from the reviewed catalogs. Additionally or alternatively, step 208 may include receiving a selection of an implant from, e.g., a user via a user device or interface. For example, the computer system may select an implant that can then be proposed to a user, who may accept or reject the proposed implant. If the user rejects the implant, or independently of a computer selecting an implant, a user may be provided with a list of implants having volumes close to the approximate implant volume, and their characteristics (e.g., on a user device). The user may then select a desired implant from the list. Optionally, a user may be able to select a magnitude of variation in volume from the approximate implant volume, and may be able to view implants falling within the selected magnitude of variation in volume or less.

Determining a target volume of secondary material based on the selected implant and the target post-operative volume according to step 210 may include subtracting the volume of the implant selected according to step 208, as well as the pre-operative volume of the implantation site, from the target post-operative volume. In other words, in determining a hybrid strategy, once an implant is selected for use in the hybrid strategy, the remaining volume of the target post-operative volume may be achieved by adding a corresponding amount of secondary material to the implantation site.

Characteristics of the secondary material may affect the extent to which it may supplement an implant at an implantation site. Thus, method 200 may then continue to step 212, which includes adjusting the target volume of secondary material based on secondary material characteristics. In this step, the volume of secondary material to be added to the implantation site may be adjusted to account for a property or behavior of the secondary material, such as an uptake rate, a reabsorption rate, and/or a survival rate. A reabsorption rate of a secondary material (e.g., a fat graft) may be, for example, between about 30% and about 60%, such as about 30%, about 35%, about 40%, about 45%, about 50%, about 55%, and about 60%. Therefore, an additional amount of the secondary material may be added to the implantation site to account for the reabsorption rate. In such cases, the total secondary material volume to be added to the implantation site may be calculated as follows:

$$Total Added Volume = Desired Volume * ( 1 + ( \frac{reabsorption rate ( \% )}{100 \%} ) ) \qquad Equation 1$$

For example, for a secondary material having a reabsorption rate of about 35%, the volume of the secondary material to be added to the implantation site may be multiplied by 1.35, such that upon expected reabsorption of a portion of the secondary material, the remaining volume of secondary material at the implantation site will be the desired volume.

Once a hybrid strategy is developed that includes both an implant and an adjusted volume of secondary material, processes may continue to, e.g., saving and/or providing a recommended surgical strategy to a user, or any other step(s).

In some embodiments, the method may be modified from that depicted in FIG. 1 and the algorithm depicted and described with respect to FIG. 2 in order to account for certain characteristics of a subject. Specifically, in some cases, applying a determined fraction or percentage to a target post-operative volume (e.g., step 206 of method 200) provides an approximate implant volume greater than the target post-operative volume. This may be true, e.g., when the target post-operative volume is less than twice the volume of the pre-operative volume at the implantation site. In some such cases, applying a determined fraction or percentage to a target post-operative volume may arrive at a supposed approximate implant volume that, when added to the pre-operative volume at the implantation site, would result in a total post-operative volume that is greater than desired. In other such cases, this step may arrive at a supposed approximate implant volume that leaves nearly no room for secondary materials. Method 300, while sharing several steps in common with method 200, includes a query and an additional step to account for such cases with a correction coefficient. Method 300 may include receiving pre-operative parameters for an implantation site (step 302), receiving a target post-operative volume for the implantation site (step 304), and applying a correction coefficient to the target post-operative volume based on a pre-operative parameter to determine an approximate implant volume (step 306). Method 300 may further include determining whether the post-operative volume is less than or equal to twice the pre-operative volume (step 308). If yes, then method 300 may include applying the correction-coefficient to the approximate implant volume to re-determine the approximate implant volume (step 310). If not, method 300 may include skipping step 310. Method 300 may then include selecting an implant based on the approximate implant volume (step 312).

Receiving pre-operative parameters for an implantation site according to step 302 may include any and/or all aspects of step 102 described with respect to method 100. Receiving a target post-operative volume for the implantation site according to step 304 may include any and/or all aspects of step 204 described with respect to method 200. Applying a correction coefficient to the target post-operative volume based on a pre-operative parameter to determine an approximate implant volume according to step 306 may share any and/or all aspects of step 206 described with respect to method 200, where the correction coefficient of step 306 is the determined fraction or percentage of the target post-operative volume that should be filled by an implant, of step 206, and wherein the pre-operative parameter is a characteristic of the subject, such as skin laxity.

According to step 308, method 300 may then include determining whether the target post-operative volume is less than or equal to twice the pre-operative volume. If not, then method 300 may continue to step 312. However, if the target post-operative volume is indeed less than or equal to twice the pre-operative volume at the target implantation site, then method 300 proceeds to step 310, which may include applying the correction coefficient to the approximate implant volume, to re-determine the approximate implant volume. This step may include multiplying the approximate implant volume by the determined fraction or percentage of the target post-operative volume that should be filled by an implant, to account for the relatively smaller overall volume difference between the pre-operative volume and the target post-operative volume.

For example, if a determined percentage of a target post-operative volume to be accounted for by an implant is 65% and the target post-operative volume is 400 cc, and the pre-operative volume of the implantation site is 200 cc (i.e., the target post-operative volume is less than or equal to twice the pre-operative volume at the target implantation site), then the approximate implant volume may be 400 cc×0.65×0.65, or 169 cc. Therefore, this step reduces the approximate implant volume to account for the fact that the overall volume difference between the pre-operative volume and the target post-operative volume is only 200 cc.

In some cases, an overall volume difference between the pre-operative volume and the target post-operative volume may be even smaller, such that applying the correction coefficient twice is insufficient. For example, a target post-operative volume may be 400 cc and the pre-operative volume of the implantation site may be 250 cc. In such a case, applying the correction coefficient twice (to arrive at an approximate implant volume of 169 cc) still results in an approximate implant volume greater than the overall volume difference between the pre-operative volume and the target post-operative volume (in this case, 150 cc). In such a case, the correction coefficient may be applied more than twice, until the approximate implant volume is less than the overall volume difference between the pre-operative volume and the target post-operative volume. Finally, selecting an implant based on the approximate implant volume according to step 312 may include any and/or all aspects of step 208 described with respect to method 200.

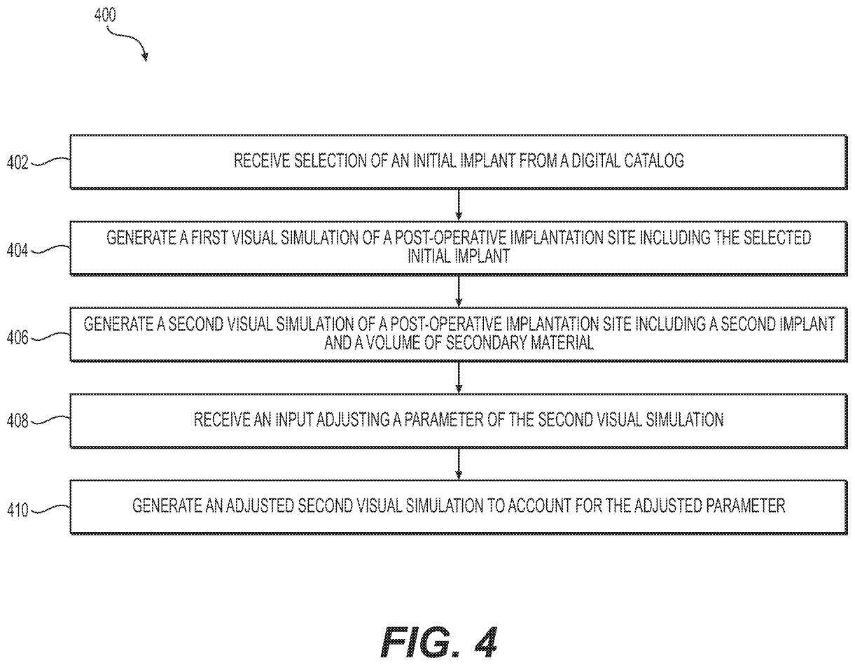

As has been alluded to in the discussion of FIGS. 1-3, aspects of methods disclosed herein may include receiving input from and/or sending output to, e.g., a user device having a user interface, which may be a visual user interface. This input and output may allow for a user (e.g., a physician, a subject, or other user) to view and change aspects of a potential surgical procedure to, e.g., achieve a more customized final result. FIG. 4 illustrates an exemplary method 400 according to aspects of the present disclosure. Method 400 may include receiving selection of an initial implant, e.g., from a digital catalog (step 402), generating a first visual simulation of a post-operative implantation site including the selected initial implant (step 404), generating a second visual simulation of a post-operative implantation site including a second implant and a volume of a secondary material (step 406), receiving an input adjusting a parameter of the second visual simulation (step 408), and generating an adjusted second visual simulation to account for the adjusted parameter (step 410).

As with the other methods disclosed herein, steps of method 400 may be performed by, e.g., a system including computing hardware and software (e.g., system 1300). In some embodiments, steps of receiving input (e.g., step 402 and step 408) may include receiving input from a user device (e.g., device 1306) and steps of generating or adjusting a visual simulation may include the use of a computer system (e.g., computer system 1304), and/or more specifically a modeling engine (e.g., modeling engine 1310). Configurations and relative locations of a user device and a computer system are described further with respect to system 1300, but in general may have any suitable configuration or location for performing the steps of method 400.

Receiving selection of an initial implant from a digital catalog according to step 402 may include, e.g., receiving identification of a particular implant in a digital catalog and/or receiving a volume, size, filling type, and/or other characteristic of an initial implant. The digital catalog may be, e.g., a tailored list or database provided according to step 106 of method 100, or may be a pre-existing list or database. In some embodiments, for example, a digital catalog may be provided to a user interface, and the user interface may subsequently receive a user selection of an initial implant.

Generating a first visual simulation of a post-operative implantation site including the selected initial implant according to step 404 may include using parameters of a pre-operative implantation site (e.g., received according to step 102 of method 100, step 202 of method 200, or step 302 of method 300) in combination with parameters of the selected initial implant to construct an image of the implantation site into which the selected initial implant has been implanted or placed. In some embodiments, generating this visual simulation may include, e.g., generating a three dimensional visual simulation using, e.g., imaging data of a subject and adjusting it to simulate the addition of the selected initial implant. Any of the three-dimensional simulations and/or related methods and algorithms disclosed in International Application No. PCT/US2019/034667 filed on May 30, 2019, incorporated by reference herein, may be used in the present disclosure.

For example, the methods herein may include generating and/or manipulating a simulation using a three-dimensional model that includes a plurality of tetrahedra used to describe tissue volume (breast tissue in a breast volume model). The simulation may be based on a three-dimensional model that corresponds to an initial configuration, wherein the model may be modified to simulate an expansion or stretching of tissue to accommodate placement of an implant and secondary materials as disclosed herein. For example, a set of reference tetrahedra of increased volume may be defined, wherein the reference tetrahedra correspond to the planned region of volume increase. Further details on generating and modifying such three-dimensional models to simulate the results of contemplated medical procedures are provided in PCT/US2019/034667 filed on May 30, 2019.

Generating a second visual simulation of a post-operative implantation site including a second implant and a volume of a secondary material according to step 406 may include, e.g., receiving or calculating a potential hybrid strategy including an implant volume (with a selected initial implant having the implant volume) and a volume of secondary material, and constructing a simulation of the hybrid strategy. In some embodiments, a visual simulation generated according to step 406 may include a standard or default distribution of the volume of the secondary material in the implantation site. In other embodiments, a visual simulation generated according to step 406 does not include the volume of the secondary material distributed at the implantation site, and may simply note an available volume of secondary material which may be added to the simulation in a customized distribution.

In some embodiments, the first visual simulation and/or the second visual simulation may be output to, e.g., a user interface. For example, a comparative view of the first visual simulation and the second visual simulation may be provided to, e.g., allow a user to view similarities and differences between the use of an initial selected implant and the use of a hybrid strategy at an implantation site. Presentation of the first visual simulation and/or the second visual simulation optionally may include interactive elements (e.g., sliders, buttons, meters, color coding, and the like) on a user interface of a device, to allow a user to change or otherwise interact with and manipulate the visual simulations.

Receiving an input adjusting a parameter of the second visual simulation according to step 408 may include receiving a change from a user through such an interactive element. This step may include receiving a change to any parameter of the second visual simulation corresponding to a change in surgical procedure or materials used. For example, a change to an implant size, an implant shape, an implant type, an implant placement position, a volume of secondary material, and/or a distribution of secondary material may be received.

Generating an adjusted second visual simulation to account for the adjusted parameter according to step 410 may include making any recalculations necessary in response to the received input to reflect the adjustment in the second visual simulation, re-generating or changing the second visual simulation according to the recalculations, and/or outputting the adjusted second visual simulation. In this manner, a user may interact with the visual simulation to observe the effect of various options and adjustments on the outcome of a surgery, and to view similarities and differences between an approach using only a selected initial implant and an approach using a second implant and a volume of secondary material (e.g., a hybrid approach).

In some embodiments, adjustments may be received for both the first and second visual simulations. In other embodiments, adjustments may be received for only the second visual simulation. Generally, method 400 may assist a user in visualizing, customizing, and otherwise preparing for a contemplated surgical procedure including an implant.

Some embodiments of the present disclosure may facilitate collaboration between a subject (e.g., a patient), and a physician (e.g., a surgeon). For example, in some embodiments of the present disclosure, a patient may be able to select a desired shape and size of a post-operative implantation site. Measurements of the patient may be performed on, e.g., a three-dimensional image of the implantation site on the client (wherein the three-dimensional image may be acquired by a camera, e.g., of a scanner), and a simulation of the patient's desired post-operative implantation site may be combined with the patient measurements to obtain a simulation of a target post-operative implantation site. Thereupon, a computer-implemented variation on surgeon selection of the breast implant may be output based upon one or more methods described herein. An algorithm may compare volumes and shapes of different implants (e.g., surface curvatures, area, etc.) to find a potential best match to a final desired shape and size. Additionally, an algorithm may rank order implants based on the initial measurements of the patient and additional geometric calculations, which may be based on physical and/or mechanical properties of the implant, physicochemical properties of the secondary materials, and/or may be based on other considerations.

Reference will now be made to views of exemplary user interfaces which may be used with aspects of the present disclosure. User interfaces suitable for combining with methods of the present disclosure may generally allow for users to view generated simulations, make selections and adjustments to them, and/or to save, load, transfer, or otherwise use the generated simulations in contemplating or preparing for a surgical procedure. As such, user interfaces according to the present disclosure may include any suitable displays, interactive elements, options, etc. to achieve these goals. The views depicted herein are merely limited examples, and one of ordinary skill in the art will understand that many more variations on exemplary user interfaces are possible and contemplated herein.

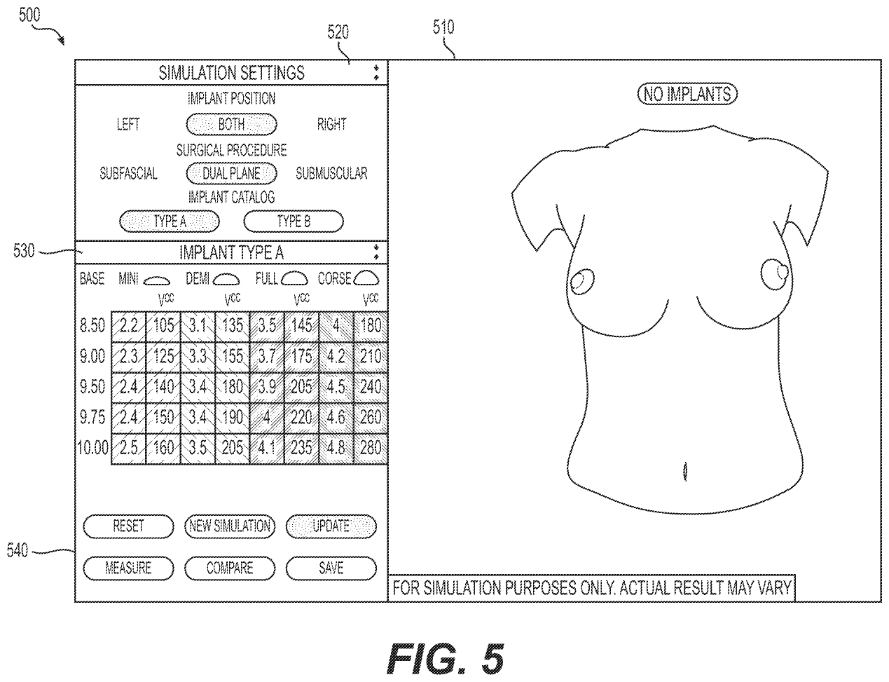

FIGS. 5-7 depict views 500, 600, 700 of an exemplary user interface for use in, e.g., simulating, modelling, designing, and preparing for aesthetic or reconstructive surgeries. View 500 may include a pre-operative visual simulation 510 of an implantation site (in this case, a torso is depicted). View 500 also includes a simulation setting menu 520 including various general simulation settings, an implant selection menu 530 listing a plurality of implants (e.g., listing dimensions of each of a plurality of implants) that may be selected and added to the simulation, and a simulation use menu 540, including options which may assist in the use of a generated simulation in various ways.

Pre-operative visual simulation 510 may be a simulation generated according to aspects of the present disclosure and any suitable method (e.g., using images, parameters, measurements, or other data pertaining to a subject, as disclosed elsewhere herein). Generally, pre-operative visual simulation 510 may depict an implantation site of a subject in a pre-operative state, i.e., before a contemplated surgical procedure to insert one or more implants to the subject's body. In some embodiments, simulation 510 may be a three dimensional simulation, such as a three-dimensional rendering. In some embodiments, simulation 510 may be interactive (e.g., rotatable, scalable, expandable, and the like). Simulation 510 may assist a user in visualizing and analyzing an initial status (e.g., size, shape, look) of an implantation site.

Simulation setting menu 520 may include one or more selectable options to customize a contemplated surgical procedure. As an example, menu 520 includes an option to select an implant position or positions, select a type of surgical procedure, and view a type of digital catalog. Types of digital catalogs may differ by, e.g., implant brand (manufacturer), implant model, shape, texture, etc. Implant selection menu 530 may include a list or other presentation of potential implants to include in the implantation site. For example, implant selection menu 530 may include a variety of dimensions (e.g., volumes, diameters, shapes, etc.). Simulation use menu 540 may include options for implementing, changing, comparing, etc. one or more implants in the simulation.

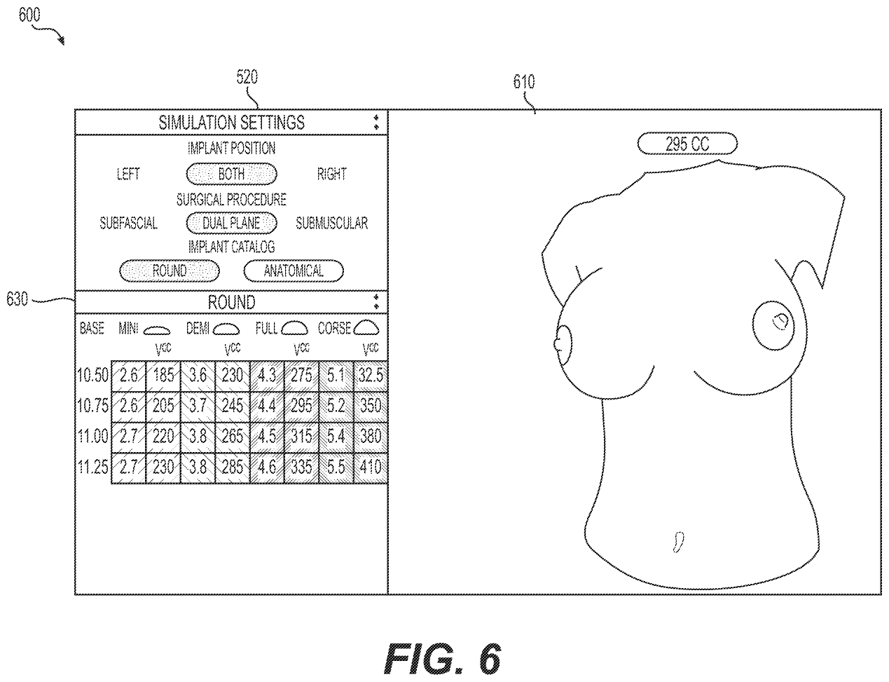

FIG. 6 depicts view 600, including a post-operative visual simulation 610 of an implantation site including an implant of a selected size. View 600 may include one or more of the same menus as view 500, such as, e.g., simulation setting menu 520, and may include one or more views which differ or vary from menus shown in view 500. For example, an exemplary implant size menu 630 is shown, which may list a variety of volumes of a single implant shape.

Post-operative visual simulation 610 may be a simulation generated according to aspects of the present disclosure and any suitable method (e.g., using images, parameters, measurements, or other data pertaining to a subject, as disclosed elsewhere herein). Generally, post-operative visual simulation 610 may depict an implantation site of a subject in a post-operative state, i.e., including one or more implants. In some embodiments, post-operative visual simulation 610 may depict a standard implantation procedure in which, e.g., an implant accounts for an entire difference in volume at an implantation site (as opposed to a hybrid strategy including an implant and a volume of a secondary material). As with simulation 510, simulation 610 may be a three dimensional simulation, such as a three-dimensional rendering, and may similarly be interactive (e.g., rotatable, scalable, expandable, and the like). Simulation 610 may assist a user in visualizing and analyzing an initial status (e.g., size, shape, look) of an implantation site.

Implant size menu 630 may be, e.g., a variation on or a sub-menu of implant selection menu 530 depicted in view 500. Implant size menu 630 may be depicted upon selection of an option to view implants having a particular shape (e.g., round implants, as opposed to anatomically shaped implants). In some embodiments, implant size menu 630 may include a smaller number of implant options than, e.g., implant selection menu 530, to allow for a user to review and select implants having a particular characteristic (in this case, a round shape). One of ordinary skill in the art will understand that many other types of implant selection menus are possible and contemplated herein.

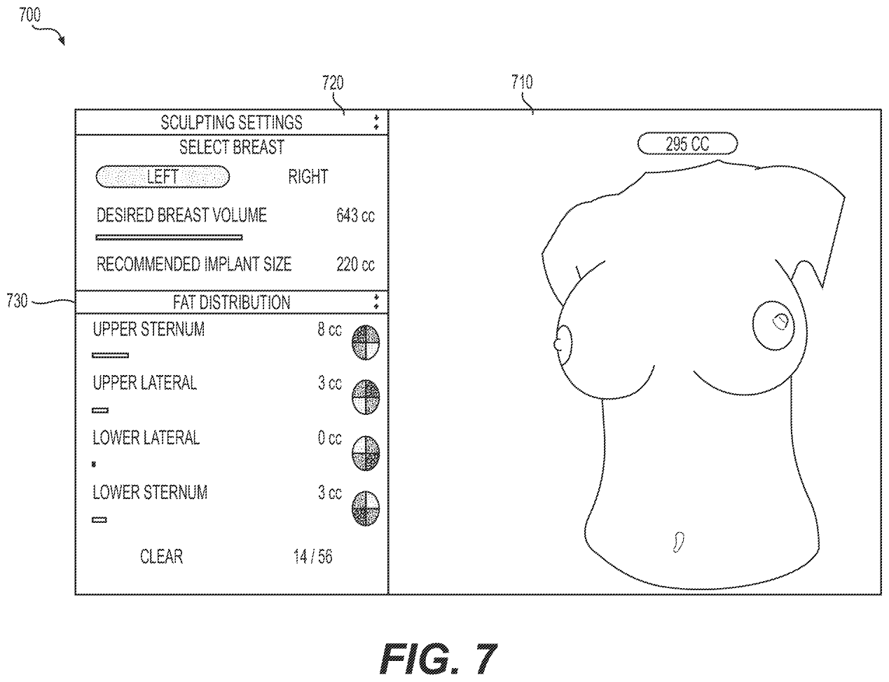

FIG. 7 depicts view 700, which may include a hybrid post-operative visual simulation 710 of an implantation site including an implant of a selected size and a volume of a secondary material (e.g., fat). View 700 may also include one or more of the same menus as views 500 and 600. View 700 may include menus specific to a hybrid post-operative simulation, such as, e.g., a sculpting settings menu 720 and a secondary material distribution menu 730.

Hybrid post-operative visual simulation 710 may be a simulation generated according to aspects of the present disclosure and any suitable method (e.g., using images, parameters, measurements, or other data pertaining to a subject, as disclosed elsewhere herein, in combination with a calculated combination of an implant and secondary material). Generally, hybrid post-operative visual simulation 710 may depict an implantation site of a subject in a post-operative state including both an implant (or two implants, in the case of a double mammoplasty as shown) and a volume of secondary material. As with simulations 510 and 610, simulation 710 may be a three dimensional simulation, such as a three-dimensional rendering, and may similarly be interactive (e.g., rotatable, scalable, expandable, and the like). Simulation 710 may assist a user in visualizing and analyzing a hybrid approach to a surgical procedure, and may allow a user to “sculpt” or otherwise alter the hybrid approach (e.g., by changing a distribution of the secondary material) to derive a customized target post-operative result.

Sculpting settings menu 720 may include options to, e.g., change an implant size or a volume of secondary material at a selected implantation site (here, a left breast or a right breast). As shown, a recommended implant size may be displayed. The recommended implant size may be calculated according to algorithms and/or methods disclosed herein (e.g., according to methods 100, 200, and/or 300). An interactive component (e.g., a slider, meter, button, or numerical input field) may allow for variation of the desired post-operative volume. In response to variation of the desired post-operative volume, a recommended implant size may change (e.g., the user interface may dynamically provide a recommended implant size based on changes to the desired post-operative volume).

Secondary material distribution menu 730 may include, e.g., division of the implantation site into sections (e.g., quadrants, as shown, or other sections) and may allow for a user to add to, subtract from, or otherwise alter the distribution of secondary material at the implantation site via interactive components (e.g., sliders, meters, buttons, numerical input fields, etc.) for each section. Algorithms may be employed to, e.g., dynamically update a simulation according to adjustments made in the secondary material distribution menu. In some embodiments, smoothing algorithms may also be employed to maintain a desirably smooth (e.g., well-integrated) look to an implantation site, regardless of changes made to the simulation in secondary material distribution menu 730. An exemplary method of updating a simulation according to changes made to secondary material distribution is described herein with respect to FIG. 12.

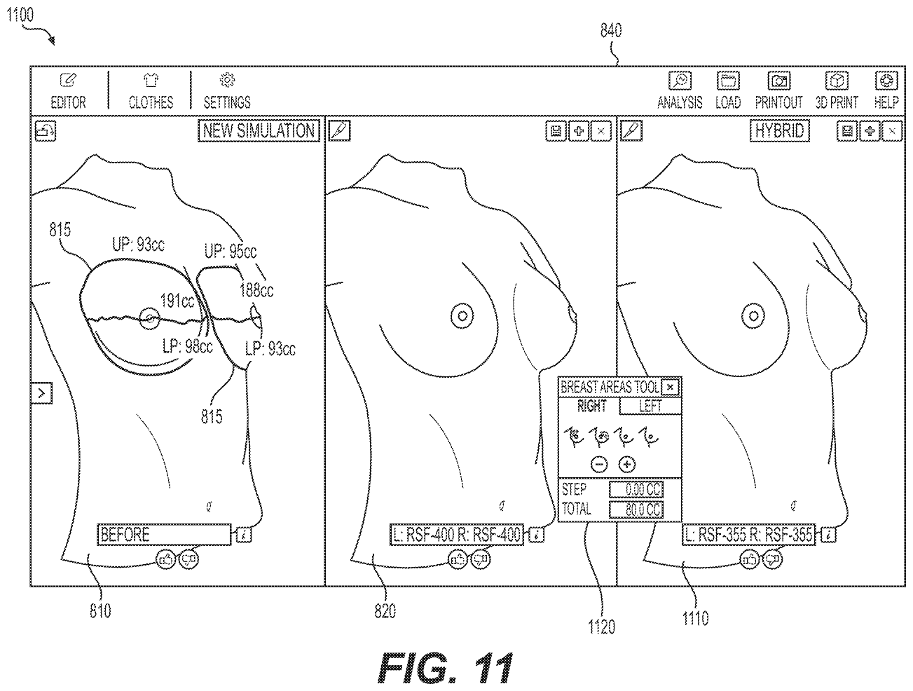

In some embodiments, views 500, 600, and 700 may all be displayable simultaneously (e.g., in separate windows). In some embodiments, in may be possible to toggle between views 500, 600, and 700, to compare the simulations shown in each view. In other embodiments, an option to directly view a side-by-side comparison of the different simulations may be available (see, e.g., FIG. 11).

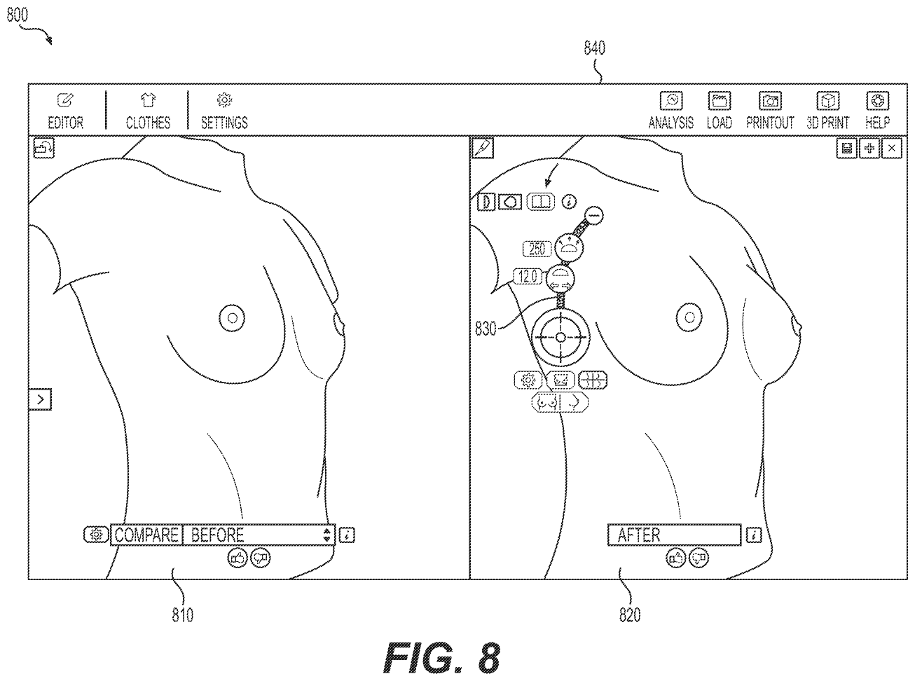

FIGS. 8-11 depict views 800, 900, 1000, 1100 of another exemplary user interface for use in, e.g., simulating, modelling, designing, and preparing for cosmetic surgeries. View 800, for example, may include a pre-operative visual simulation 810 of an implantation site, and a comparative post-operative visual simulation 820 of the implantation site. A post-operative visual simulation adjustment menu 830 may allow for changes to post-operative visual simulation 820. Simulation use menu 840 may include options to assist in the use of generated simulations in various ways.

Pre-operative visual simulation 810 may share characteristics with, e.g., pre-operative visual simulation 510 of view 500. Post-operative visual simulation 820 may likewise share characteristics with, e.g., post-operative visual simulation 610 of view 600. View 800 allows for the pre- and post-operative visual simulations to be viewed simultaneously for a more direct comparison between the two. In some embodiments, pre-operative visual simulation 810 and post-operative visual simulation 820 may both be rotatable, expandable, or otherwise viewable in tandem, so that similar views of the two simulations may be examined simultaneously.

Adjustment menu 830 may be an interactive element or collection of interactive elements allowing a user to adjust post-operative visual simulation 820. In some embodiments, post-operative visual simulation 820 may dynamically change depending on adjustments made in adjustment menu 830. Adjustment menu 830 may include options to change, e.g., post-operative volume, shape, height, projection, or other characteristics of an implantation site. Additionally, post-operative visual simulation 820 and/or adjustment menu 830 may show numeric characteristics of, e.g., an implantation site in post-operative visual simulation 820. For example, a post-operative volume and/or other dimension of an implantation site may be dynamically calculated as post-operative visual simulation 820 is adjusted, by calculating, e.g., differences between pre-operative visual simulation 810 and post-operative visual simulation 820 in real time or periodically. Thus, a post-operative volume and/or diameter (and/or other dimensions) of an implantation site may be indicated as a part of post-operative visual simulation 820 and/or adjustment menu 830. In this manner, view 800 may allow a user to adjust and view characteristics of a post-operative implantation site until a desirable post-operative implantation site is achieved. In some embodiments, arrival at a desirable post-operative implantation site via, e.g., adjustments made using adjustment menu 830 may also serve as a selection of a post-operative volume.

Simulation use menu 840 may include various selectable options to aid in use of the simulation(s). For example, simulation use menu 840 may include options to load, print, save, analyze, annotate, or visually present a simulation or simulations. Simulation use menu 840 may be available on, e.g., multiple views of a user interface to allow for saving, loading, and otherwise manipulating simulations from any of the multiple views.

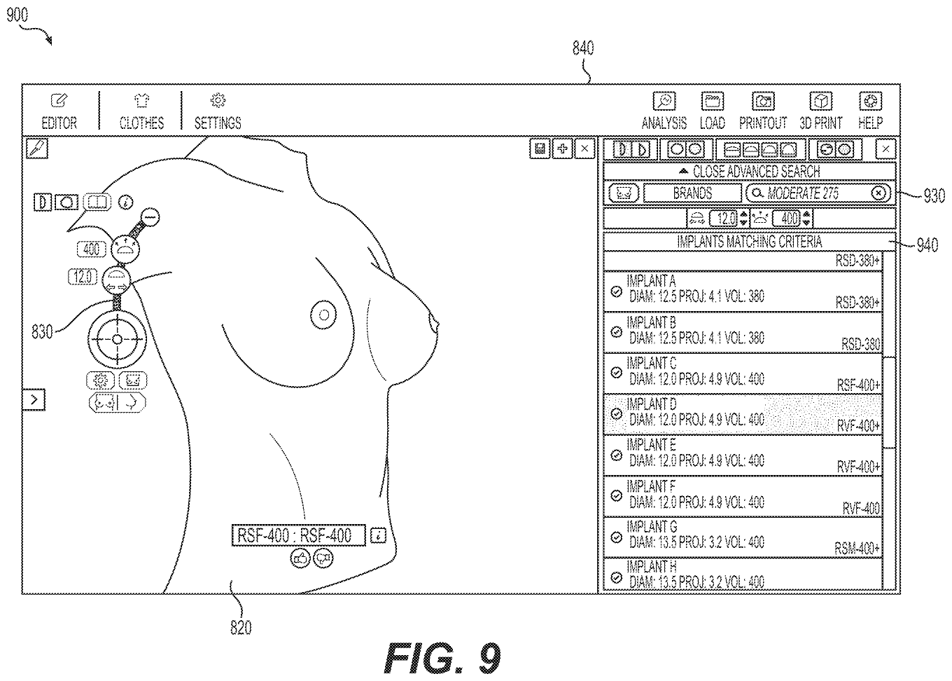

FIG. 9 depicts view 900, which may include post-operative visual simulation 820, adjustment menu 830 an implant search menu 930, and an implant selection menu 940. View 900 may include one or more of the same menus as view 800, such as simulation use menu 840.

Implant search menu 930 may accept input from a user to populate implant selection menu 940 with a list of implants compatible with characteristics of post-operative visual simulation 820. In some embodiments, for example, implant search menu 930 may accept input of search criteria, such as a particular implant brand, shape, or having a particular texture or filling type. Implant selection menu 940 may list implants fitting the search criteria that also have measurements that may be suitable for achieving the post-operative visual simulation from, e.g., a pre-operative implantation site. Some implants in implant selection menu 940 may be identified as hybrid-compatible implants—i.e., they may be used in combination with a volume of a secondary material, as a part of a hybrid strategy. A user may then be able to select an implant from implant selection menu 940 for further refinement of the post-operative visual simulation 820.

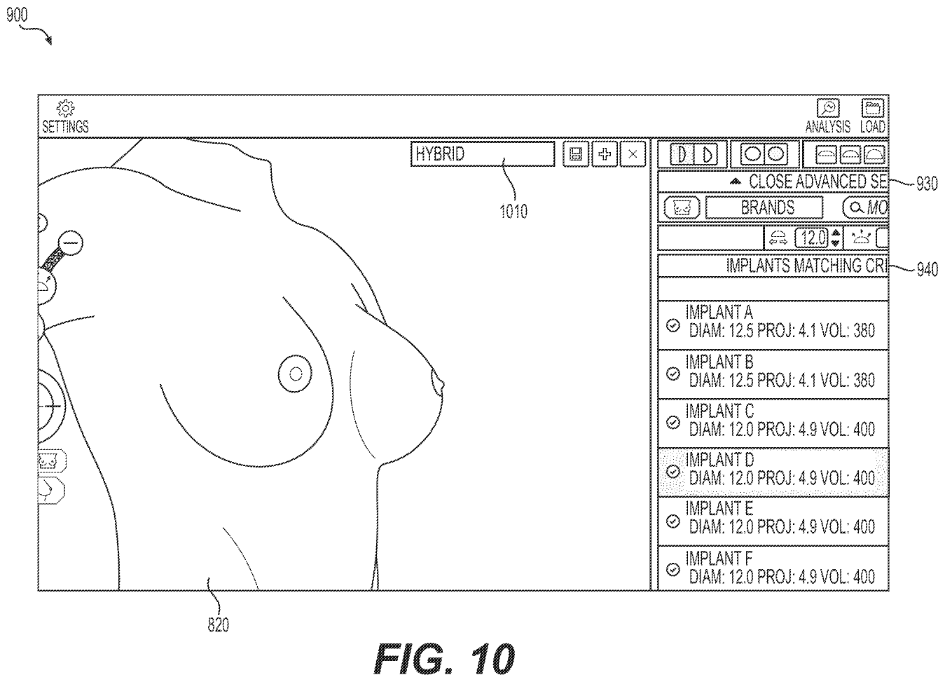

FIG. 10 depicts a close-up portion of view 900, in which a hybrid-compatible implant has been selected from implant selection menu 940. Upon selection of a hybrid-compatible implant, a button 1010 allowing for the generation of a hybrid strategy may be digitally added to view 900. Button 1010 may generate a new post-operative hybrid simulation, as depicted in FIG. 11.

FIG. 11 depicts view 1100, which may include a comparison of several simulations, such as pre-operative visual simulation 810, post-operative visual simulation 820, and a hybrid post-operative visual simulation 1110. View 1100 may include an adjustment tool 1120, which may allow a user to adjust parameters of, e.g., post-operative visual simulation 920 and/or hybrid post-operative visual simulation 1110. Moreover, view 1100 depicts measurement lines 815, which may be applied by a user or digitally to identify different sections of an implantation site. While measurement lines 815 are depicted on pre-operative visual simulation 815, similar measurement lines may be applied to post-operative visual simulation 820 or hybrid post-operative visual simulation 1110.

Hybrid post-operative visual simulation 1110 may share any or all characteristics with, e.g., hybrid post-operative visual simulation 710 of view 700. Generally, hybrid post-operative visual simulation 1110 may depict an implantation site of a subject in a post-operative state including both an implant based on a selection made from implant selection menu 940 and a volume of secondary material. As with other simulations disclosed herein, simulation 1110 may be a three dimensional simulation, such as a three-dimensional rendering, and may similarly be interactive (e.g., rotatable, scalable, expandable, and the like). Simulation 1110 may assist a user in visualizing and analyzing a hybrid approach to a surgical procedure. Adjustment tool 1120 may allow a user to input commands to “sculpt” or otherwise alter the hybrid approach (e.g., by changing a volume, distribution, or type of the secondary material) to derive a customized target post-operative result.

Methods for simulating the addition of a volume of secondary material to an implantation site may benefit from, e.g., algorithms that may automatically apportion the secondary material in a realistic and suitable manner around an implant/implantation site. Such algorithms may be run as a part of, e.g., a modeling engine (see, e.g., modeling engine 1310 of system 1300). Generally, algorithms may help a modeling engine to simulate the addition of secondary materials into (or removal of secondary materials from) one or more segments of an implantation site, and may advantageously allow for greater control and customization in the simulation of secondary materials at the implantation site. For example, segmentation of an implantation site may provide greater precision in parameters for placement of the secondary materials at the implantation site. Segments of an implantation site may be digitally determined once, e.g., a perimeter and/or a center or epicenter of an implantation site are identified. For example, for a mammoplasty, a simulated breast area may be digitally separated into four quadrants which may intersect at a point of greatest projection or at a nipple portion of the breast.

In simulating the addition or subtraction of secondary materials at a segment of an implantation site, methods according to the present disclosure and algorithms for implementing such methods may address two goals: simulating a realistic increase (or decrease) in rest volume of one or more digital segments of an implantation site, and realistically contouring the perimeter of such segments and/or the implantation site, so that a smooth transition between the area inside of a segment and the area outside of a segment (e.g., either within the implantation site or external to the implantation site) is maintained.

To achieve both of these goals, segments of an implantation site may be further divided into geometric subsections (e.g., tetrahedra or other suitable shapes). In some embodiments, for example, a tetrahedral model of an implantation site (e.g., a breast model) may be constructed from a triangle mesh surface. Each subsection may be identified or characterized by a distance between the sub-section (e.g., a center point of the subsection) and a reference point (e.g., a center or epicenter of an implantation site, a point on a perimeter of an implantation site, or both). Segments may be divided into small enough subsections to allow for realistic modeling, without overtaxing processing power of, e.g., a modeling engine.

In some embodiments, segments and/or subsections may be selected and oriented based on the general location of an implantation site. For example, in the case of a breast implantation site, a chest wall of a subject may be used to orient a coordinate system for dividing the implantation site into segments and/or subsections. A rotation matrix may be formed that rotates center points of subsections (e.g., tetrahedral center points) from a global space to an orientation of the chest wall. Points in the coordinate system may be translated so that a nipple location is located at the origin of the coordinate system. Each of the four quadrants of the coordinate system may then be translated into one of four segments that make up the breast implantation site (e.g., such that points where x<0 and y<0 correspond to a southwest quadrant, points where x>0 and y<0 correspond to a southeast quadrant, points where x<0 and y>0 correspond to a northwest quadrant, and points where x>0 and y>0 correspond to a northeast quadrant).

To simulate an increase in volume in a segment of an implantation site, a modeling engine may increase a volume of subsections in the segment by some fraction of their pre-operative or otherwise original volume. A scaling factor for the volume increase may be computed from the original volume of the segment, and from the volume of added secondary material. The volume increase may be tapered on a subsection-by-subsection basis, depending on the characterization of each subsection, such that a greater volume increase is modeled at a center of a segment than at a periphery of the segment. This may allow for smoother transitions between segments. Moreover, the volume increase may be tapered depending on additional reference points, such as a body surface of the subject, a direction of gravitational pull, and the like.

To simulate a realistic/smooth contour at a perimeter of an implantation site, a modeling engine may separate a border area of a segment into a plurality of points, and may apply a displacement to each point depending on a distance between the point and the nearest point of a surface outside of the segment (e.g., a perimeter point outside of the implantation site). The border area may be determined, e.g., algorithmically, or may be determined using user input. The magnitude of displacement for each point of the plurality of points may negatively correlate to a distance between the segment and the point. For example, a fraction of a displacement towards a perimeter point may be applied to each point within border area, where the fraction decays to zero as the distance between each point and the nearest perimeter point decreases. An exemplary function that has been implemented to achieve this is:

$$f = {( 1 - \frac{d}{{D}_{max}} )}^{e} \qquad Equation 2$$

where d is a distance to the nearest perimeter point, Dₘₐₓ is a width of the border area (i.e., d≤Dₘₐₓ), and e is a decay exponent parameter that may be chosen.

FIG. 12 illustrates an exemplary perimeter contouring method 1200 according to aspects of the present disclosure. Method 1200 may include receiving data regarding a volume of material to add to an implantation site (step 1202), separating the implantation site into regions (step 1204), separating each region into sub-regions (step 1206), simulating addition of the volume of material to a region of the implantation site by increasing a volume of each sub-region in the region, wherein the increase in volume for each sub-region is dependent on a distance between the sub-region and an origin point (step 1208), and performing a smoothing algorithm to simulate a smooth change in volume between the region of the implant site and an area outside of the region (step 1210).

Receiving data regarding a volume of material to add to an implantation site according to step 1202 may include receiving, e.g., a manually-input or automatically-suggested volume of a secondary material to add to an implantation site. Separating the implantation site into regions according to step 1204 may be done as described above, e.g., by identifying quadrants or other segments of the implantation site based on a center point, an epicenter, or a border of the implantation site. Separating each region into sub-regions according to step 1206 may also be done as described above, e.g., by separating each segment into subsections and characterizing each subsection by a center point and a distance between the center point and one or more reference points (e.g., a center, epicenter, or periphery of a segment or of the implantation site). Simulating addition of the volume of material to a region of the implantation site according to step 1208 may include identifying subsections lying within the region (e.g., the segment) in question, and increasing their reference volumes. For example, if a total starting volume of subsections in a region or segment is characterized by V₀, and of is a volume of a secondary material to be added to the region or segment, then the reference volume of each subsection in the region or segment may increase by a factor of s:

s=(V₀+vf)/V₀ Equation 3

In some embodiments, a modeling engine may apply this or a similar algorithm to a segment or region and will move towards a solution in that segment or region in which the subsections are a factor of s larger than their initial sizes. As described above, a volume added to each subsection may taper in correlation with a distance between a center point of the subsection and a perimeter or other reference point of the region or segment. Performing a smoothing algorithm to simulate a smooth change in volume between the region of the implant site and an area outside of the region according to step 1210 may include applying Equation 2 to, e.g., points in a border area of the region.

Embodiments disclosed herein may be created, executed, displayed etc. using any suitable technology or system. In some embodiments, methods disclosed herein may be executed using one or more computer systems. FIG. 13 depicts a high-level schematic diagram of an exemplary system 1300 for executing the methods described herein (e.g., methods 100, 200, 300, 400, 1200). Specifically, FIG. 13 depicts an imaging system 1302, computer system 1304, and user device 1306, all of which may be connected to an electronic network 1320.

System 1300 is merely exemplary, and it may be understood by one of ordinary skill in the art that system 1300 may have any configuration suitable for facilitating execution, display etc. of embodiments disclosed herein. In some embodiments, components of system 1300 (e.g., imaging system 1302, computer system 1304, and user device 1306) may be part of a unitary device or system. In other embodiments, components of system 1300 may each include a separate device, computer, or group of computers.

Imaging system 1302 may be any imaging system suitable for, e.g., receiving imaging data characterizing an implantation site. As has been described elsewhere herein, imaging data may include two- or three-dimensional images, physical measurements, or any other data suitable for creating a simulation of, and/or analyzing, an implantation site. In some embodiments, imaging system 1302 may include one or more image capture devices designed to capture two- and three-dimensional images of anatomy, such as cameras, scanners (e.g., including one or more cameras), x-ray devices, computerized tomography devices, magnetic resonance imaging devices, positron emission tomography devices, and/or other devices. In some embodiments, imaging system 1302 may include a scanner specifically designed to obtain detailed visual data to aid in simulating an implantation site. For example, imaging system 1302 may include aspects of systems disclosed in International Application Publication No. WO2017/175055, incorporated by reference herein in its entirety. Imaging system 1302 may be located, e.g., in a medical facility, office, educational facility, home, or any other suitable location. In some embodiments, imaging system 1302 may be portable. In some embodiments, it is contemplated that system 1300 may include multiple types of imaging systems 1302.

Computer system 1304 may include one or more computers configured to process, store, create, and/or manipulate images and data received from, e.g., imaging system 1302 and/or user device 1306. In some embodiments, computer system 1304 may be combined with imaging system 1302 and/or user device 1306 into a single device or system. In other embodiments, computer system 1304 may receive and/or send data over network 1320 to and/or from imaging system 1302 and/or user device 1306. Computer system 1304 may include, for example, a storage module 1312 for storing images and data, and a processing module 1308 for processing and manipulating images and data. Computer system 1304 may also include a modeling engine 1310 which may create and/or update simulations using images and data received from imaging system 1302 and/or user device 1306, and/or stored in storage module 1312. Computer system 1304 may also include, for example, a recommendations engine 1314 which may generate and/or update one or more suggestions or recommendations of parameters for a surgical procedure (e.g., an implant size and/or a volume of secondary material). In some embodiments, modeling engine 1310 and recommendations engine 1314 may, together or separately, receive and send data and/or perform aspects of the methods disclosed herein (e.g., methods 100, 200, 300, 400, 1200).

Storage module 1312 may include one or more computers, computer processors, hard drives, cloud-based storage systems, and/or other system configured to electronically store data and/or images. Storage module 1312 may store received data in, for example, one or more databases, digital file systems, and/or cloud-based storage systems. Further, computer system 1304 may process received data in processing module 1308. Processing module 1308 may process data by, for example, cataloguing received data, sorting data by one or more categories, such as by patient, anatomical feature, intervention, measurement device, date created, date received, etc. Such processing may also include analyzing data and/or selecting data to send to modeling engine 1310 and/or recommendations engine 1314. Storage module 1312 and/or processing module 1308 may send received data, before or after it is stored and/or processed, to modeling engine 1310 and/or recommendations engine 1314.

Storage module 1312, processing module 1308, modeling engine 1310, and recommendations engine 1314 may each or all be, for example, one or more computer processors, computer storage devices, and/or combinations thereof. Modeling engine 1310 may generate and/or update a simulation (e.g., a three-dimensional visual simulation) according to embodiments of the present disclosure e.g., a three-dimensional simulation using relevant data received from storage module 1312 and/or processing module 1308. Recommendations engine 1314 may run one or more algorithms to generate one or more recommended strategies for surgical procedures (e.g., hybrid surgical procedures) according to aspects of embodiments disclosed herein, based at least in part on data received from storage module 1312 processing module 1308, and/or a simulation created or updated by modeling engine 1310.

Modeling engine 1310 and/or recommendation engine 1314 may be located on any hardware capable of allowing them to perform the functions described herein. For example, modeling engine 1310 and recommendation engine 1314 may operate on a single computer, or may be a set of networked computers, working, for example, in series or in parallel. In further embodiments, functions of modeling engine 1310 and recommendation engine 1314 may be shared by two computational machines, or four or more computational machines.

User device 1306 may be any device suitable for facilitating user interaction with a simulation of an implantation site—e.g., creating, modifying, or viewing a simulation of a pre-operative or post-operative implantation site in order to create, customize, or otherwise prepare for a surgical procedure including an implant. User device 1306 may include, for example, a device allowing for output of a simulation, suggestions, and/or recommendations for a surgical procedure to a user. User device 1306 may also allow for input of subject data, implant data, secondary material data, parameters, measurements, and/or adjustments to measurements for creating or modifying a simulation. A user may be, e.g., a physician, a patient, a prospective patient, or any other individual. In some embodiments user device 1306 may be a computing device, such as, e.g., a personal computer or a computer associated with a medical facility, a tablet, or a mobile device. Generally, user device 1306 may be any computing device having a graphical user interface.

Network 1320 may be, for example, a wired or wireless network of computer processors, electronic storage devices, etc., such as the Internet, a local area network, a wide area network, or any other computer network configuration known in the art. In some embodiments, network 1320 may be entirely local to a single geographic area, e.g., a single medical facility, office, or server system. In other embodiments, network 1320 may connect various aspects of system 1300 across different geographic areas, e.g., different medical facilities, offices, cities, countries, or continents.

The following examples are intended to illustrate the present disclosure without, however, being limiting in nature. It is understood that the present disclosure encompasses additional embodiments consistent with the foregoing description and following examples.

### EXAMPLES

### Example 1

A patient has a pre-operative breast volume of 150 cc, and the patient's surgeon has chosen 400 cc implants. Based on the patient's skin laxity, a desired volume of an implant as compared to total breast volume is 65% (i.e., the desired implant volume is 65% of the total desired breast volume, based on the skin laxity of the patient). A computer system runs an algorithm which makes the following determinations:
150 cc+400 cc=550 cc (total desired breast volume)
500 cc (total desired breast volume)×0.65 (coefficient based on the desired implant volume)=357.5 cc (total desired implant volume, given skin laxity of the patient).

The implant having the nearest available volume below the 550 cc desired implant volume is a 350 cc implant. The computer system includes this implant as part of a hybrid strategy recommendation. The computer system then determines the remaining volume by subtracting the implant volume from the total desired breast volume and the existing patient tissue (pre-operative breast volume).
550 cc (total desired breast volume)−350 cc (volume of available implant)−150 cc (pre-operative breast volume)=50 cc (calculated volume of secondary material).

The secondary material selected is autologous fat, having a reabsorption rate of 35% or 0.35. Thus, the computer system calculates the volume of secondary material based on the reabsorption rate as follows: 50 cc×1.35 (to account for predicted reabsorption)=67 cc (recommended volume of fat). The computer system includes this volume as a part of the hybrid strategy recommendation.

Thus, where a surgeon originally chooses 400 cc implants, the algorithm recommends a hybrid approach of a 350 cc implant and 77 cc of autologous fat. This hybrid approach provides the patient with a more naturalistic look and feel.

### Example 2

A patient has a pre-operative breast volume of 200 cc, and the patient's surgeon has chosen 200 cc implants. Based on the patient's skin laxity, a desired implant volume as compared to total breast volume is 65% (i.e., the desired implant volume is 65% of the total desired breast volume, given skin laxity of the patient). A computer system runs an algorithm which makes the following determinations:
200 cc+200 cc=400 cc (total desired breast volume)
400 cc (total desired breast volume)×0.65 (coefficient based on the desired implant volume)=260 cc.

As the total desired breast volume is less than or equal to twice the patient's pre-operative breast volume, the coefficient is applied again:
260 cc×0.65=169 cc (total desired implant volume, given skin laxity of the patient).

The implant having the nearest available volume below the desired implant volume is a 155 cc implant. The computer system includes this implant as part of a hybrid strategy recommendation. The computer system then determines the remaining volume by subtracting the implant volume from the total desired breast volume and the existing patient tissue (pre-operative breast volume).
400 cc (total desired breast volume)−155 cc (volume of available implant)−200 cc (pre-operative breast volume)=45 cc (calculated volume of secondary material).

The secondary material selected is autologous fat, having a reabsorption rate of 50% or 0.5. Thus, the computer system calculates the volume of secondary material based on the reabsorption rate as follows: 45 cc×1.5 (to account for predicted reabsorption)=67.5 cc (recommended volume of fat). The computer system includes this volume as a part of the hybrid strategy recommendation.

Thus, where a surgeon originally chooses 200 cc implants, the algorithm recommends a hybrid approach of a 133 cc implant and about 67 cc of autologous fat.

While the figures and disclosure herein depict several exemplary configurations of transponders, sensors, assemblies, readers, implants, and several exemplary methods of use thereof, one of ordinary skill in the art will understand that many other configurations and variations on methods are possible and may be appropriate for a given implant, patient, procedure, or application, based on implant size, shape, orientation and intended location in the patient body. The examples of devices, systems, and methods herein are intended to be exemplary and are not comprehensive; one of ordinary skill in the art will also understand that some variations on the disclosed devices, systems, and methods herein are also contemplated within this disclosure.

1. A method for simulating an outcome of a surgical procedure, the method comprising:
receiving parameters for a pre-operative state of an implantation site;
receiving parameters for a post-operative state of the implantation site;
based on the parameters for the pre-operative and post-operative states, automatically generating a hybrid strategy for achieving the post-operative state from the pre-operative state, wherein the hybrid strategy includes a proposed implant volume and a proposed volume of a secondary material; and
generating a simulation of the post-operative state of the implantation site using the proposed implant volume and the proposed volume of the secondary material.

2. The method of claim 1, wherein receiving parameters for the post-operative state of the implantation site comprises:
providing a catalog of potential implants for use at the implantation site; and
receiving a selection of an initial implant from the catalog of potential implants.

3. The method of claim 1, wherein the parameters for the pre-operative state of the implantation site include a pre-operative volume, and wherein the parameters for the post-operative state of the implantation site include a post-operative volume.

4. The method of claim 1, wherein automatically generating the hybrid strategy comprises:
calculating a difference in volume between the post-operative state and the pre-operative state;
determining the proposed implant volume by applying a skin quality coefficient to the difference in volume; and
determining the proposed volume of the secondary material by subtracting the proposed implant volume from the difference in volume.

5. The method of claim 4, wherein a volume of the post-operative state is less than or equal to twice a volume of the pre-operative state, and wherein determining the proposed implant volume further comprises:
applying the skin quality coefficient to the volume difference to obtain a first value; and
applying the skin quality coefficient to the first value to obtain the proposed implant volume.

6. The method of claim 5, wherein the skin quality coefficient is between about 0.5 and about 0.7.

7. The method of claim 4, wherein determining the proposed volume of the secondary material further comprises factoring in a reabsorption rate of the secondary material.

8. The method of claim 1, wherein the secondary material comprises fat or a synthetic filler.

9. A method for simulating an outcome of a surgical procedure, the method comprising:
receiving parameters for a pre-operative state of an implantation site;
receiving a selection of an initial implant from a digital catalog;
generating a first visual simulation of the implantation site in a first post-operative state, wherein the first post-operative state includes the selected initial implant;
generating a second visual simulation of the implantation site in a second post-operative state, wherein the second post-operative state includes a second implant, optionally selected from a digital catalog or database, and a volume of secondary material;
receiving an input adjusting a parameter of the second post-operative state; and
generating an adjusted second visual simulation of the implantation site in the second post-operative state to account for the adjusted parameter.

10. The method of claim 9, wherein the adjusted parameter includes a change in a distribution of the volume of secondary material at the implantation site.

11. The method of claim 9, wherein the input adjusting the parameter of the second post-operative state includes a third implant different from each of the initial implant and the second implant.

12. The method of claim 9, wherein the steps of receiving parameters for a pre-operative state of an implantation site, receiving selection of an initial implant from a digital catalog, and receiving an input adjusting a parameter of the second post-operative state include receiving data from a graphical user interface.

13. The method of claim 9, wherein the second visual simulation includes a distribution of the volume of secondary material in one or more quadrants of the implantation site, and wherein receiving the input adjusting the parameter of the second post-operative state includes receiving an indicated amount of secondary material for addition to or subtraction from at least one quadrant of the one or more quadrants of the implantation site.

14. The method of claim 9, further comprising:
displaying a side-by-side view of the first visual simulation and the second visual simulation.

15. The method of claim 9, wherein receiving parameters for the pre-operative state of the implantation site includes receiving digital imaging data of the implantation site.

16. A method for simulating an outcome of a surgical procedure, the method comprising:
receiving parameters for a pre-operative state of an implantation site;
generating a simulation of the implantation site using the parameters for the pre-operative state and a proposed implant volume;
receiving placement parameters for a volume of secondary material to be added to the implantation site; and
modifying the simulation of the implantation site to include the volume of secondary material.

17. The method of claim 16, wherein generating the simulation of the implantation site includes dividing the implantation site into segments.

18. The method of claim 16, wherein the placement parameters for the volume of secondary material comprises identification of a segment for placement of the volume of secondary material.

19. The method of claim 18, wherein modifying the simulation of the implantation site comprises:
increasing a volume of the identified segment corresponding to the volume of secondary material; and
generating a resulting displacement at a plurality of points on a perimeter of the implantation site, wherein a magnitude of displacement for each point of the plurality of points negatively correlates to a distance between the identified segment and the point.

20. The method of claim 16, wherein the proposed implant volume corresponds to an implant from a catalog of potential implants, and/or wherein the secondary material comprises fat or a synthetic filler.
